(19) United States
(12) Patent Application Publication
(10) Pub. No.: US 2005/0186186 A1
(43) Pub. Date: Aug. 25, 2005

(54) Compositions for eliciting immune response and methods for using same

(76) Inventors: Jiangping Wu, Brossard (CA); Guixiu Shi, Saranac Lake, NY (US)

Correspondence Address: GOUDREAU GAGE DUBUC, 800 PLACE VICTORIA, SUITE 3400, MONTREAL, QUEBEC, H4Z 1E9, CA

(21) Appl. No.: 11/045,357

(22) Filed: Jan. 31, 2005

Related U.S. Application Data
(60) Provisional application No. 60/539,966, filed on Jan. 30, 2004.

(51) Int. Cl. A61K 48/00; C12N 5/08

(52) U.S. Cl. 424/93.21; 435/366

(57) ABSTRACT

A tumor cell having DcR3/TR6 anchored at its surface, a composition comprising same and methods comprising same which may advantageously be used to elicit an immune response in a patient in need thereof.

### CROSS REFERENCE TO RELATED APPLICATIONS

This application claims priority on U.S. provisional application No. 60/539,366, filed on Jan. 30, 2004 which is herein incorporated by reference.

### FIELD OF THE INVENTION

The present invention relates to compositions for eliciting immune response and methods for using same. It relates more specifically to anti-tumor compositions for eliciting immune response and methods for using same.

### BACKGROUND OF THE INVENTION

TR6/DcR3. TR6, a new member of the TNFR family, has 3 known ligands: FasL, TL1A and LIGHT (1-3). In humans, TR6 is a secreted protein (1, 4). We carried out BLAST search of mouse genome sequences with human TR6 as a query, but no significant match was found, indicating that the mouse does not have a counterpart of human TR6. In the immune system, TR6 mRNA is expressed at high levels in lymph nodes and the spleen (1, 5), while its expression in the thymus and peripheral blood lymphocytes is weak or undetectable.

TR6 can bind to FasL and inhibit the interaction between Fas and FasL. Consequently, FasL-induced apoptosis in lymphocytes and several tumour cell lines can be inhibited by TR6 (1). Theoretically, TR6 should be able to interfere with Fas-mediated T-cell costimulation (6). Interaction between TR6 and TL1A disrupts costimulation by TL1A through its receptor DR3, and results in abated T-cell responses (2). TR6 also inhibits TL1A-induced apoptosis of DR3-expressing erytheroleukemic TF-1 cells (2). Human TR6 can bind to both human and mouse LIGHT. It can probably block LIGHT-induced apoptosis. TR6 can also bind to both human and mouse FasL (1-3, 7). This feature allows human TR6 to be function in mouse models.

LIGHT is a new member of the TNF family (8), and its protein is expressed on activated T cells (8) and immature dendritic cells (9). We have demonstrated that resting T cells also express a considerable amount of LIGHT on their surface, but it is better detected by confocal microscopy than by flow cytometry (10). LIGHT is a ligand for HveA and LTPR, both of which are TNFR members (8). HveA is constitutively expressed at both protein and mRNA levels in most lymphocyte subpopulations, including CD4 and CD8 T cells (11,12). LIGHT can induce apoptosis in cells expressing both HveA and LTPR (13), but Rooney et al. (14) showed that LTβR is necessary and sufficient for LIGHT-triggered apoptosis in tumour cells. Since LTβR is not expressed on lymphocytes (15), LIGHT is unlikely to cause apoptosis in these cells.

Recent studies show that LIGHT can costimulate T-cell responses via HveA in vitro and in vivo (9,11,12,16). Moreover, transgenic mice overexpressing LIGHT have augmented immune responses (17), and LIGHT knockout (KO) mice present defects in cytotoxic T cell activity (18,19). Taken together, these lines of evidence indicate that LIGHT functions as a costimulating molecule via HveA for T-cell activation.

Reverse signalling through LIGHT. Although being ligands, several TNF members on cell surfaces can reversely transduce signals into T cells. Cayabyab (20) and van Essen (21) demonstrated that CD40L could transduce costimulation signals into T cells. Wiley reported that CD30L crosslinking can activate neutrophils (22), and Cerutti found that such reverse signalling inhibits Ig class switch in B cells (23). Reverse signalling through membrane TNF-α confers resistance of monocytes and macrophages to LPS (24). Crosslinking of TRANCE enhances IFN-γ secretion by activated Th1 cells (25). Reverse signaling through FasL can promote maximal proliferation of CD8 cytotoxic T cells (26-28).

We have reported that LIGHT can also transduce signals reversely into T cells (10,29). Solid phase TR6-Fc significantly augmented mouse CD4 and CD8 cell proliferation under suboptimal TCR stimulation. Under such a condition, IL2 and IFN-g secretion was enhanced in CD4 cells, and IFN-g but not IL2 secretion was increased in CD8 cells. Similarly, solid phase TR6-Fc stimulated human T-cell proliferation and lymphokine production. Although solid phase TR6 stimulated Th1 and Th2 cell proliferation equally well, it preferentially enhanced IFN-γ production in TH1 cells but not IL5 production in Th2 cells, suggesting that costimulation via LIGHT reverse signaling is more important in Th1-type immune responses. Consistent with this notion, solid phase TR6-Fc enhanced cytotoxic T-cell (CTL) activity in both humans and mice. It should be noted that the Fc in the recombinant TR6-Fc has been mutated so that it no longer binds to FcγRs; any possible effect of TR6-Fc via FcγR has thus been ruled out.

The following evidence collectively proves that a part of the effect of solid phase TR6-Fc, as described above, occurs via LIGHT on T cells. 1) Soluble LIGHT blocked TR6 binding to Th1 and Th2 cells; TR6 bound to about 82% wild-type T cells, but only 18% LIGHT KO T cells, indicating that LIGHT represents a significant TR6-binding partner on T cells. 2) More importantly, mAb against LIGHT, like TR6, when put on solid phase, could also stimulate T-cell proliferation. With these new findings on LIGHT reverse signaling, the results from LIGHT transgenic mice and knockout mice can be reinterpreted. The increased LIGHT reverse signaling might contribute to the augmented immune responses observed in LIGHT transgenic mice; conversely, elimination of such reverse signaling might contribute to the abated immune responses seen in LIGHT knockout mice. Such reinterpretation does not refute the importance of forward LIGHT costimulation mediated by HveA. As TR6 can bind to FasL and FasL can also reversely transduce signals into T cells (26-28), it is possible that TR6 on the solid phase can trigger T cell costimulation via both LIGHT and FasL.

We have further demonstrated that after T-cell activation, LIGHT rapidly co-congregated with TCR, and both TCR and LIGHT were translocated to rafts. This provides a morphological basis for the signaling pathways of LIGHT and TCR to interact, and allows LIGHT to access the abundant signaling molecules located in the raft scaffold. We have also shown that p44/42 MAPK was activated after LIGHT crosslinking, and such activation was a necessary signaling event for costimulation via LIGHT reverse signaling, because a p44/42 MAPK-specific inhibitor repressed the costimulation. All these pieces of evidence on LIGHT reverse signaling have been published in our two recent articles (10,29).

The present description refers to a number of documents, the content of which is herein incorporated by reference in their entirety.

### SUMMARY OF THE INVENTION

This invention concerns the discovery that when DcR3/TR6 is anchored on tumor cell surface, it costimulates tumor antigen-specific T cells, enhances tumor immunogenicity and consequently, contributes to treat and/or prevent tumors.

As used herein the term “anchored” in the expression “cell having DcR3/TR6 anchored at its surface” refers to any attachment of the DcR3/TR6 that enables the protein to elicit an immune response in the host un which the cell is introduced. Without being so limited, it includes the DcR3/TR6 being anchored to the cell through a heterologous transmembrane, the transmembrane domain being recombinantly attached to the DcR3/TR6. It also includes methods using bifunctional chemicals, or biotin/streptavidin to link DcR3/TR6 to any cell surface protein.

With regards to the use of a transmembrane domain or membrane anchoring peptide for anchoring DcR3/TR6 to the cell surface, a person of ordinary skill in the art will understand that although in the illustrative examples presented herein, the coding sequence for the transmembrane domain of EphB6 was used (accession number: NM_{—}007680), the coding sequence of the transmembrane domain of any transmembrane protein could be used in accordance with the present invention. Furthermore, any peptide of 10 to 30 amino acids which mainly comprise hydrophobic amino acids could be used as membrane anchoring peptide in accordance with the present invention. Similarly, although in the examples disclosed herein, a specific transfection vector was used for expressing the recombinant DcR3/TR6 protein, a person of ordinary skill in the art would understand that other expression systems, such as other transfection vectors, electroporation, adenovirus, adenovirus-associated virus, retrovirus could be used to express the recombinant molecules on any tumor cell surface. Also a person of ordinary skill in the art would understand the promoter desirably used in the present invention is one that enables a high level of expression of the protein that it drives. See also Fussenegger M. et al, “Genetic optimization of recombinant glycoprotein production by mammalian cells” for known method to produce recombinant protein Tibtech, January 1999 (Vol. 17) for examples of known methods for recombinant protein expression.

As used herein the terminology “growth inhibition” when applied to a cell refers to any treatment applied to this cell to prevent its proliferation. A cell so treated is then “growth inhibited”. Without being so limited, such treatment includes subjecting the cell to chemicals able to prevent proliferation such as an antineoplastic agent, or a hormone antagonist or agonist for tumors sensitive to these agents, or a cytokine (such as IL-2 as an example) for tumors sensitive to it, or an immunotoxin which is an toxin-conjugated antibody specific to a tumor, irradiation, heating, freezing, or a combination of two or more of these treatments.

As used herein the terminology “an immune eliciting fragment of a tumor cell” refers to a fragment of the cell to which is anchored a DcR3/TR6. Indeed, it is not necessary to introduce intact cells in the patients for the immune response to be desirably elicited. Indeed, a membrane fragment bearing a DcR3/TR6 is sufficient to elicit the desired response in the patients.

Tumor cells introduced into the patient in accordance with the present invention may be isolated. They can also be part of a tumor mass which may include not only tumor cells but also normal cells. The cells used may be from the patient in which they are introduced or from a different patient or a combination of both. The tumors could be freshly isolated or have been stored at a low temperature from a previous surgery.

The present compositions, methods and uses can be applied to any patient in need of antitumor prophylaxic or therapeutic treatment.

The quantity of cells or fragments to be administered may be as low as one and as high as 10¹⁰. The route of introduction/administration of the cells may be any suitable route including intravenous, s.c., i.m., i.p., or directly into tumors. For each treatment, the cells or fragments may be introduced once, more than once and up to 999 times.

The inoculation to a patient in need thereof of DcR3/TR6-expressing cells or fragment thereof can be performed before the patient has undergone complete or partial tumor resection, after that procedure, or even both before and after tumor resection. Of cause the inoculation can be done to a patient who has not and will not undergo tumor resection.

More specifically, in accordance with the present invention, there is provided a tumor cell having DcR3/TR6 anchored at its surface. In a specific embodiment, the tumor cell was transfected or transduced to express DcR3/TR6 at its surface. In other specific embodiments, the cell is malignant or benign. In a other embodiment, the cell is growth inhibited. In a other more specific embodiment, the growth inhibition is achieved through a treatment selected from the group consisting of a chemical treatment, irradiation, heating, freezing, and a combination thereof. In a other embodiment, there is provided an immune eliciting fragment of a tumor cell according to the present invention.

There is also provided a composition comprising tumor cells according to the present invention. There is also provided a composition comprising fragments of tumor cells according to the present invention. In an other embodiment, the composition further comprises an adjuvant. In an other more specific embodiment, the adjuvant is BCG.

There is also provided a recombinant vector which comprises in sequence a DNA sequence encoding a suitable promoter driving the expression of a DNA sequence encoding DcR3/TR6, and of a DNA sequence encoding a membrane anchoring peptide, and a poly A signal.

There is also provided a method of eliciting an immune response in a patient in need thereof, comprising introducing into the patient a composition comprising tumor cells having DcR3/TR6 anchored at their surfaces or immune eliciting fragments of the cells. In an other embodiment, the method further comprises introducing a further dose of the composition to the patient. In an other embodiment, the method further comprises simultaneously administrating a further immune therapy to the patient. In this method the patient is concurrently submitted to a further immune therapy which in a more specific embodiment is selected from the group consisting of chemotherapy, radiotherapy, hormonal therapy, or a combination thereof. In an other embodiment, the composition further comprises an adjuvant. In an other embodiment, the adjuvant is BCG.

There is also provided a method to inhibit the development of a tumor in a patient in need thereof, which comprises the steps of: obtaining a cell population from a tumor mass or tissue susceptible to tumor development; having this cell population express DcR3/TR6 on the cell membrane surface thereby obtaining a modified cell population, a fragmented cell preparation or a cell fraction comprising membrane DcR3/TR6; and administering said modified cell population, preparation or fraction to the patient so as to elicit a stronger immune reaction towards said tumor. In an other embodiment, the modified cell population is obtained by transfecting the cell population with a nucleic acid comprising a coding sequence of DcR3/TR6 linked to a coding sequence of a membrane anchoring peptide DNA coding sequence.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 schematically illustrates a construct to express membrane-bound TR6. The full-length TR6 cDNA followed by the EphB6 transmembrane domain coding for E591-R621 and then followed by a stop codon was cloned into a vector pAdenoVator (Qbiogene). The GFP coding sequence is after the IRES (internal ribosome entry segment);

FIG. 2 graphically illustrates that TR6 expression on tumor cell surface costimulates T-cell proliferation. Mitomycin C-treated surface TR6-expressing 293 cells (293-TR6), or P815 cells (P815-TR6) were used to stimulate human or mouse T cell, respectively, at 1:1 ratio (0.8×10⁶ cells/well/200 ul) in 96-well plates. Vector-transfected 293 cells (293-C) or P815 cells (P815-C), and wild type 293 cells and P815 cells were used as controls. For mouse T cell culture, a suboptimal concentration of soluble anti-CD3 (2C11 at 20 ng/ml) was present. The cells were pulsed with ³H-thymidine for 16 h before being harvested on days as indicated;

FIG. 3 graphically illustrates that surface TR6 expressed on 293 cells or P815 cells augment lymphokine production. Human (upper three panels) or mouse (lower three panels) T cells were stimulated with surface TR6-expressing 293 cells (293-TR6) or P815 cells (P815-TR6), as described in FIG. 2. The cell supernatants were harvested on days as indicated, and IFN-γ, IL2 and IL4 were measured with ELISA. Vector-transfected 293 cells (293-C) and P815 cells (P815-C), and wild type 293 cells and P815 cells were used as controls;

FIG. 4 graphically illustrates that P815-TR6 and P815 cells have similar growth rate in vitro. 5×10⁴ P815-TR6 cells and wild type P815 cells were culture in 10 ml medium. The cultures were sampled every day for cell concentration with flow cytometry. The total cell number in the culture from day 0 to day 4 is plotted;

FIG. 5 graphically illustrates the reduced tumorigenicity of P815 cells expressing surface TR6. 5×10⁴ surface TR6-expressing P815 cells (P815-TR6), vector-transfected P815 cells (P815-C) or wild type P815 cells were inoculated s.c. into the left flank of DBA/2 mice. Tumor size was measured with a caliper q.2d for 30 days and is recorded with a value which equals to the longest diameter times shortest diameter. Tumor size of mice succumbed to tumor load was assigned as 400 mm²;

FIG. 6 graphically illustrates that P815-TR6 tumor cell immunization protects parental p815 cell challenge. DBA-2 mice were first immunized with 1×10⁶ mitomycin C-treated wild type P815 cells (p815), control vector-transfected p815 cells (p815C) or TR6 vector transfected p815 cells (p815-TR6) once a week for 2 times. The mice were challenged with 5×10⁴ wild type p815 cells. The tumor size was measured as described in FIG. 5. Numbers 1 to 8 refer to mouse numbers;

FIG. 7 graphically illustrates that P815 cells expressing surface TR6 were effective as therapeutic tumor vaccine. 5×10⁴ live wild type P815 cells were inoculated s.c. into the left flank of DBA/2 mice. On days 3 and 8, 5×10⁶ mitomycin-C-treated P815-TR6 cells were inoculated on the right flank of the mice as therapeutic vaccine. Tumor size was recorded q. 2d for 30 days and is plotted; and

FIG. 8 graphically illustrates that B16 cells expressing surface TR6 were effective alone or in combination with adjuvant BCG as therapeutic tumor vaccine. 5×10⁴ live wild type low antigenic B16 cells were inoculated s.c. into the left flank of syngeneic C57BL/6 mice. On days 3 and 8, 5×10⁶ mitomycin-C-treated B16-TR6 cells which were stably transfected with surface TR6-expressing vector, or B16-C cells, which were transfected with a control vector, or wild type B16 cells were mixed with 0.5 mg BCG, were inoculated on the right flank of the mice as therapeutic vaccine. Tumor size was recorded q. 2d for 17 days and is plotted. Numbers 1 to 10 refer to mouse numbers.

### DESCRIPTION OF ILLUSTRATIVE EMBODIMENTS

This invention will be described herein below, by reference to specific examples, embodiments and figures, the purpose of which is to illustrate the invention rather than to limit its scope.

Although the present invention has been described hereinabove by way of specific embodiments thereof, it can be modified, without departing from the spirit and nature of the subject invention as defined in the appended claims.

### EXAMPLE 1

### To Express the Normally Soluble Protein DcR3/TR6 on Cell Surface

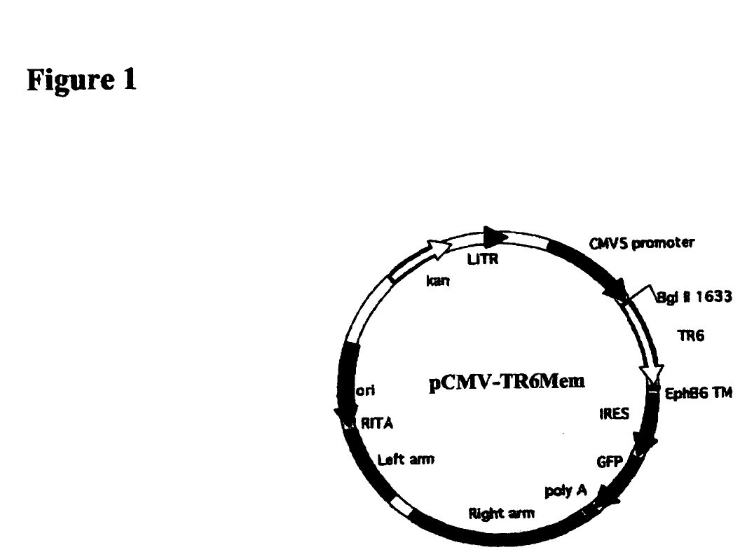

DcR3/TR6, although a member of the TNF receptor family, lacks the transmembrane domain in its coding sequence. In order to express this molecule on the cell surface, we connect the TR6 coding sequence with a coding sequence of a transmembrane domain of a molecule EphB6. The construct is illustrated in FIG. 1. The construct is co-transfected with pcDNA3 at 10:1 ratio, using Lipofectamine, into mouse P815 and human 293 cells. The transfected cells were selected with G418 for 3 weeks.

### EXAMPLE 2

### The Tumor Cells Expressing Surface DcR3/TR6 Have Enhanced Antigenicity In Vitro

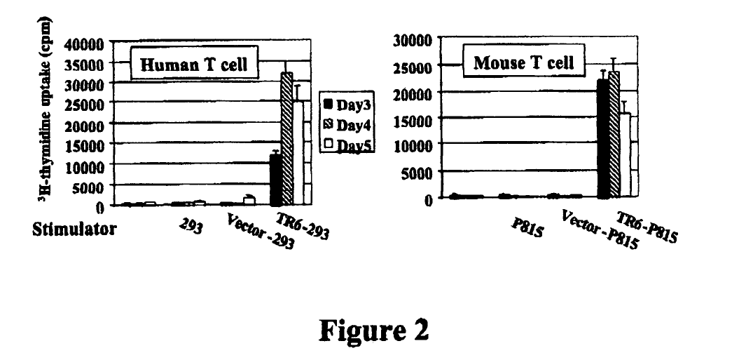

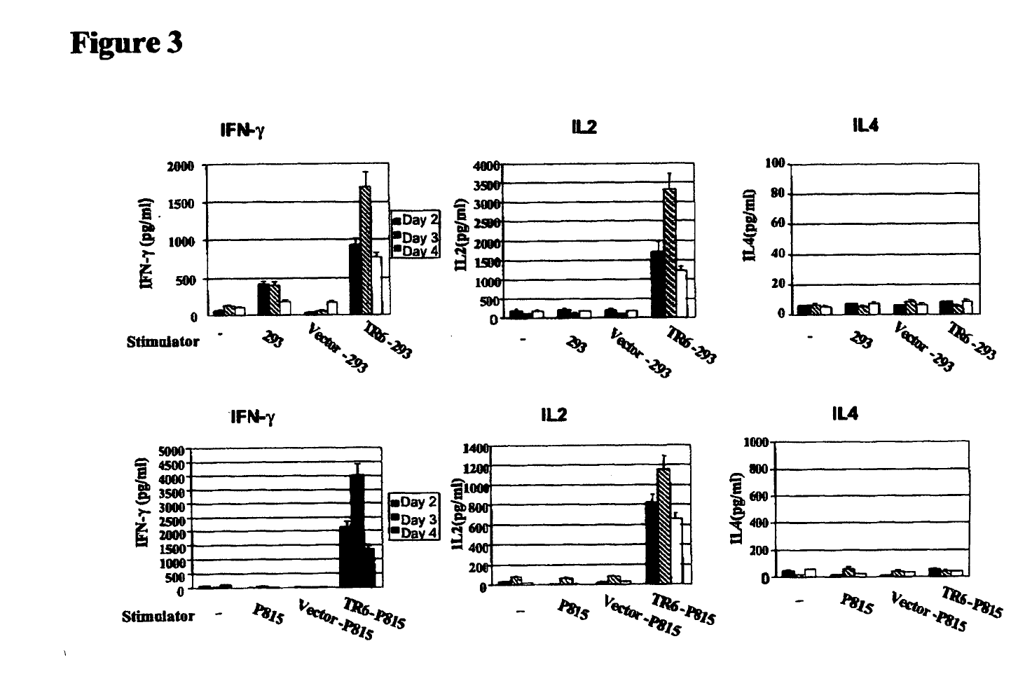

To illustrate that the surface TR6 can costimulate T cells, we inactivated the surface TR6 expressing mouse P815 cells (P815-TR6) and human 293 cells (293-TR6) with an antineoplastic agent, namely mitomycin C, and used these cells as stimulators to stimulate BALB/c spleen cells and human peripheral blood mononuclear cells (PBMC), respectively. For the former combination, a minute amount of anti-CD3 (0.01 mg/ml for coating) was coated on solid phase to enhance the first signal through TCR. In both cases, wild type tumor cells or vector-transfected tumor cells failed to stimulate T cell proliferation, while P815-TR6 and 293-TR6 cells vigorously did (FIG. 2). We also assessed cytokine production in this in vitro model, and showed that P815-TR6 and 293-TR6 could greatly enhance IL-2 and IFN-γ production (FIG. 3). These results clearly show that tumor cell surface expression of DcR3/TR6 can enhance tumor cell immunogenicity in vitro.

### EXAMPLE 3

### Tumor Cells Expressing Surface TR6 Failed to Develop into Solid Tumors In Vivo

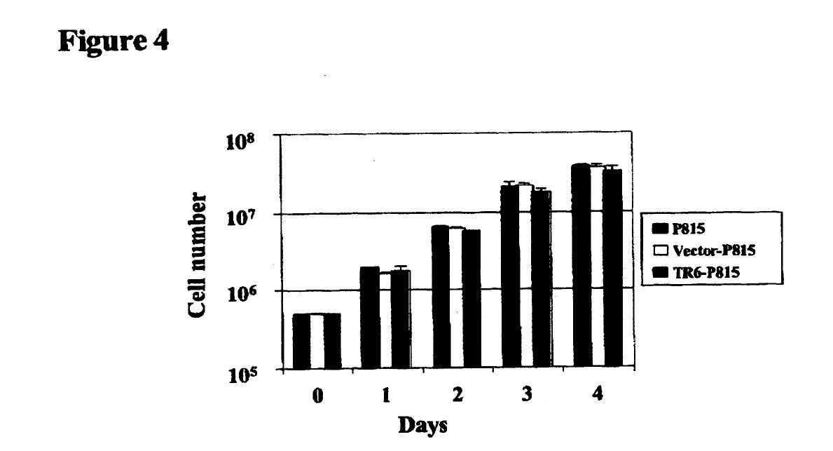

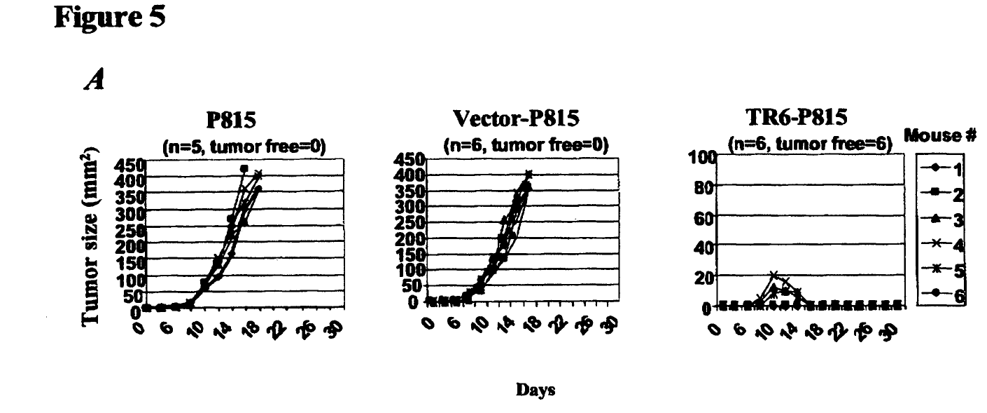

As tumors expressing TR6 on surface had enhanced antigenicity in vitro, we next assessed whether such tumor cells could be more efficiently eliminated by the host immune system in vivo. For this purpose, we first established that wild type P815 and P815-TR6 cells had similar growth rates in vitro (FIG. 4). This excluded the possibility that any difference in their speed to form solid tumors in vivo was due to different rates of tumor growth. When wild type P815, vector-transfected P815 (P815-C) and TR6-expressing P815 (P815-TR6) were inoculated into syngeneic DBA mice, the former two types readily formed tumors, while the last-mentioned group failed to do so (FIG. 5). The difference between the P815-TR6 group versus wild type P815 group, and the P815-TR6 group versus P815-C group are highly significant (One way analysis of variance, p<0.001). This result indicates that when tumor cells express surface TR6, they effectively trigger tumor immune response of the host, and this leads to they own elimination.

### EXAMPLE 4

### Mice Immunized with TR6-Expressing Tumor Cells were Resistant to Subsequent Tumor Challenge

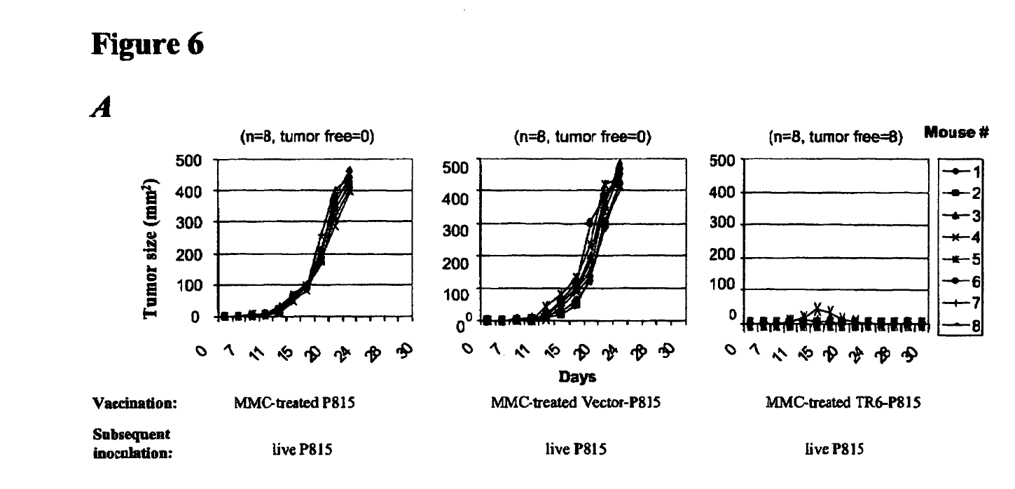

We next evaluated whether TR6-expressing tumors could be used as tumor vaccine. For this purpose, P815-TR6 tumor cells were inactivated with mitomycin C and injected s.c. into syngeneic DBA mice as vaccine. Such vaccination was conducted twice at a one-week interval. Seven days after the second vaccination, live wild type P815 cells were inoculated on the collateral flank. As shown in FIG. 6, mice vaccinated with control cells (i.e., inactivated wild type P815 or P815-C) still developed tumors, while mice vaccinated with P815-TR6 did not. The difference is highly significant (one way analysis of variance, p<0.001). This clearly indicates that surface expression of TR6 on tumor cells can trigger tumor immunity, which eliminates the subsequently inoculated tumors.

### EXAMPLE 5

### Tumor Cells Expressing Surface TR6 Could be Used as Therapeutic Vaccine

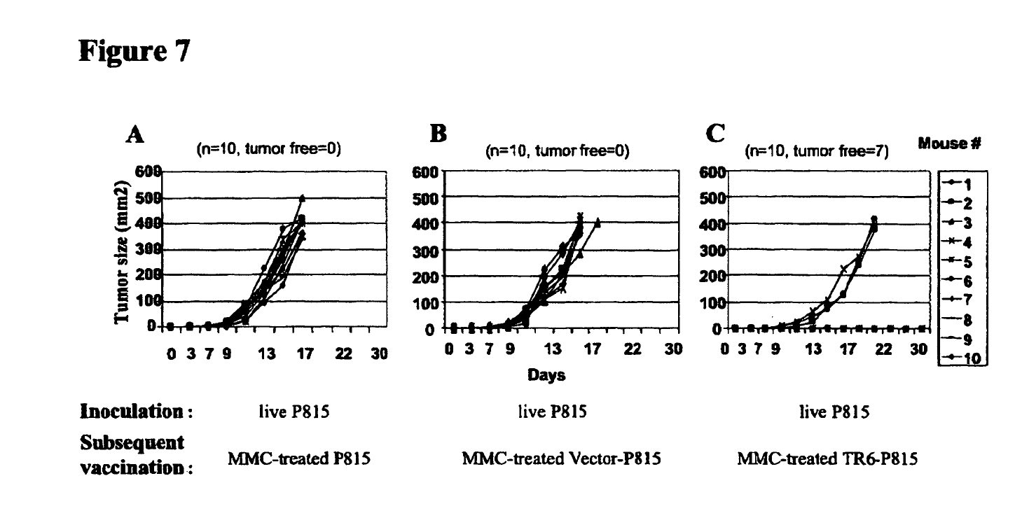

In clinical situations, patients needing tumor vaccine normally already have existing tumors in their body, and an effective vaccine should be able to eliminate existing tumor cells in the patients. To evaluate the usefulness of our approach in such a situation, we inoculated live P815 tumors into DBA mice. Three days later, these mice were vaccinated with inactivated P815-TR6 cells at a one-week interval. As shown in FIG. 7, only mice vaccinated with P815-TR6 cells, but not control cells such as wild type P815 or P815-C, could prevent tumor development in 7 out of 10 mice. The difference is highly significant (one way analysis of variance, p<0.001). This result indicates that in a clinical situation, if one takes the tumor cells from a tumor patient, let it express surface TR6, and then apply such manipulated and inactivated tumor cells as vaccine to the patient, one could achieve therapeutic effect for the patients by eliminating or slowing down the growth of the existing tumors cells in the patients.

### EXAMPLE 6

### The Therapeutic Effect of TR6-Expressing Tumor Vaccine can be Enhanced by Simultaneous Administration of Immune Adjuvant

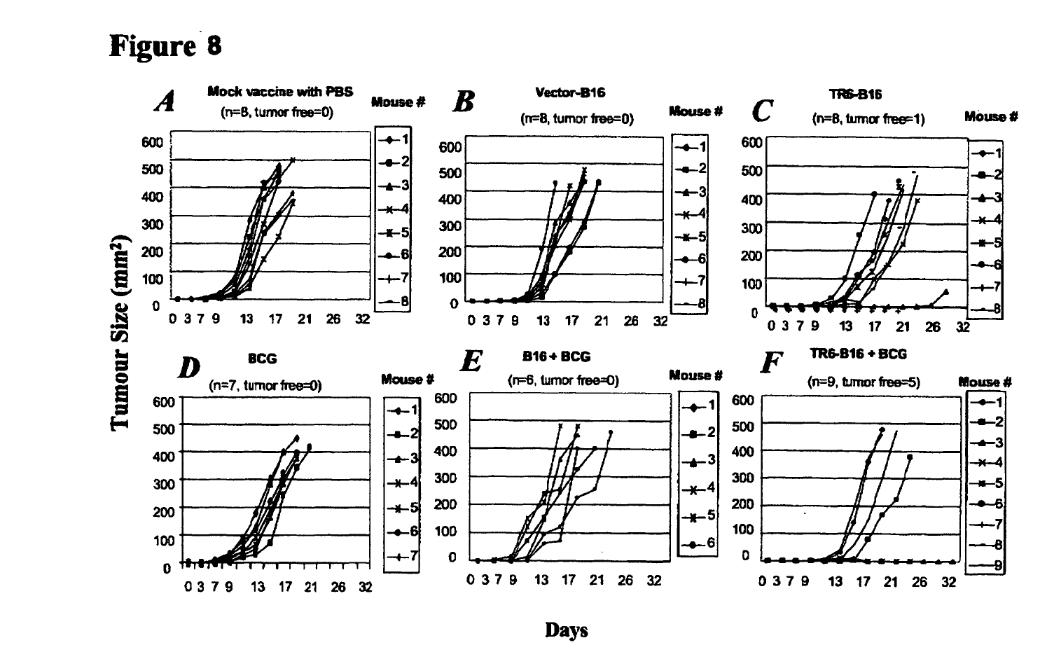

Most tumors in humans are of low antigenicity. To prove that vaccine using TR6 surface expression on tumor cells can have therapeutic effect for human tumors, we selected a low antigenic tumor B16, which is derived from a melanoma, and transfected B16 cells with the surface TR6-expressing plasmid. Wild type B16 cells and B16-C cells (B16 cells transfected with the control vector) were used as controls. As shown in FIG. 8, B16-TR6 immunization after the inoculation of live B16 tumor cells in syngeneic C57BL/6 mice reduced tumor incidence and rates of tumor growth, compared with mice vaccinated with B16-C or B16. Moreover, we also observed that when the cell vaccine was administrated along with the adjuvant BCG, the therapeutic effect was more effective in terms of further reduced tumor incidence and tumor growth rates. Thus, TR6-expressing tumor cells can be used as an effective therapeutic vaccine for tumors of low antigenicity, and the effect of such vaccine can be enhanced by simultaneous administration of other immune therapy such as BCG.

The invention being hereinabove described, it will be obvious that the same be varied in many ways. Those skilled in the art recognize that other and further changes and modifications may be made thereto without departing from the spirit of the invention, and it is intended that all such changes and modifications fall within the scope of the invention, as defined in the appended claims.

### REFERENCES

1. Pitti, R. M., S. A. Marsters, D. A. Lawrence, M. Roy, F. C. Kischkel, P. Dowd, A. Huang, C. J. Donahue, S. W. Sherwood, D. T. Baldwin, P. J. Godowski, W. I. Wood, A. L. Gurney, K. J. Hillan, R. L. Cohen, A. D. Goddard, D. Botstein, and A. Ashkenazi. Genomic amplification of a decoy receptor for Fas ligand in lung and colon cancer. Nature 1998. 396:699
2. Migone T S, Zhang J, Luo X, Zhuang L, Chen C, Hu B, Hong J S, Perry J W, Chen S F, Zhou J X, Cho Y H, Ullrich S, Kanakaraj P, Carrell J, Boyd E, Olsen H S, Hu G, Pukac L, Liu D, Ni J, Kim S, Gentz R, Feng P, Moore P A, Ruben S M, Wei P. TL1A is a TNF-like ligand for DR3 and TR6/DcR3 and functions as a T cell costimulator. Immunity 2002. 16:479-492
3. Yu K Y, Kwon B, Ni J, Zhai Y, Ebner R, Kwon B S. A newly identified member of tumor necrosis factor receptor superfamily (TR6) suppresses LIGHT-mediated apoptosis. J. Biol. Chem. 1999. 274:13733-13736.
4. Zhang J, Salcedo T W, Wan X, et al. Modulation of T-cell responses to alloantigens by TR6/DcR3. J. Clin. Invest. 2001. 107:1459-1468.
5. Bai, C., B. Connolly, M. L. Metzker, C. A. Hilliard, X. Liu, V. Sandig, A. Soderman, S. M. Galloway, Q. Liu, C. P. Austin, and C. T. Caskey. Overexpression of M68/DcR3 in human gastrointestinal tract tumors independent of gene amplification and its location in a four-gene cluster. Proc. Natl. Acad. Sci. 2000. 97:1230
6. Siegel R M, Chan F K, Chun H J, Lenardo M J. The multifaceted role of Fas signaling in immune cell homeostasis and autoimmunity. Nat Immunol. 2000. 1:469-474.
7. Wu, Y., Han, B., Luo, H., Roduit, R., Zhang, J., and Wu, J. DcR3/TR6 Effectively Prevents Islet Primary Nonfunction after Transplantation. Diabetes. 2003. 52: 2279-2286.
8. Mauri, D. N., R. Ebner, R. I. Montgomery, K. D. Kochel, T. C. Cheung, G. L. Yu, S. Ruben, M. Murphy, R. J. Eisenberg, G. H. Cohen, P. G. Spear, and C. F. Ware. LIGHT, a new member of the TNF superfamily, and lymphotoxin alpha are ligands for herpesvirus entry mediator. Immunity. 1998. 8:21.
9. Tamada, K., K. Shimozaki, A. I. Chapoval, Y. Zhai, J. Su, S. F. Chen, S. L. Hsieh, S. Nagata, J. Ni, and L. Chen. LIGHT, a TNF-like molecule, costimulates T cell proliferation and is required for dendritic cell-mediated allogeneic T cell response. J. Immunol. 2000. 164:4105.
10. Shi, G., Luo, H., Wan, X., Salcedo, T. W., Zhang, J., and Wu, J. Mouse T cells receive costimulatory signals from LIGHT, a TNF family member. Blood, 2002. 100: 3279-3286.
11. Harrop, J. A., M. Reddy, K. Dede, M. Brigham-Burke, S. Lyn, K. B. Tan, C. Silverman, C. Eichman, R. DiPrinzio, J. Spampanato, T. Porter, S. Holmes, P. R. Young, and A. Truneh. Antibodies to TR2 (herpesvirus entry mediator), a new member of the TNF receptor superfamily, block T cell proliferation, expression of activation markers, and production of cytokines. J. Immunol. 1998. 161:1786.
12. Kwon, B. S., K. B. Tan, J. Ni, K. O. Oh, Z. H. Lee, K. K. Kim, Y. J. Kim, S. Wang, R. Gentz, G. L. Yu, J. Harrop, S. D. Lyn, C. Silverman, T. G. Porter, A. Truneh, and P. R. Young. A newly identified member of the tumor necrosis factor receptor superfamily with a wide tissue distribution and involvement in lymphocyte activation. J. Biol. Chem. 1997. 272:14272.
13. Zhai, Y., R. Guo, T. L. Hsu, G. L. Yu, J. Ni, B. S. Kwon, G. W. Jiang, J. Lu, J. Tan, M. Ugustus, K. Carter, L. Rojas, F. Zhu, C. Lincoln, G. Endress, L. Xing, S. Wang, K. O. Oh, R. Gentz, S. Ruben, M. E. Lippman, S. L. Hsieh, and D. Yang. LIGHT, a novel ligand for lymphotoxin beta receptor and TR2/HVEM induces apoptosis and suppresses in vivo tumor formation via gene transfer. J. Clin. Invest 1998. 102:1142
14. Rooney, I. A., K. D. Butrovich, A. A. Glass, S. Borboroglu, C. A. Benedict, J. C. Whitbeck, G. H. Cohen, R. J. Eisenberg, and C. F. Ware. The lymphotoxin-beta receptor is necessary and sufficient for LIGHT-mediated apoptosis of tumor cells. J. Biol. Chem. 2000. 275:14307
15. Browning, J. L., I. D. Sizing, P. Lawton, P. R. Bourdon, P. D. Rennert, G. R. Majeau, C. M. Ambrose, C. Hession, K. Miatkowski, D. A. Griffiths, A. Ngamek, W. Meier, C. D. Benjamin, and P. S. Hochman. Characterization of lymphotoxin-alpha beta complexes on the surface of mouse lymphocytes. J. Immunol. 1997. 159:3288
16. Tamada, K., K. Shimozaki, A. I. Chapoval, G. Zhu, G. Sica, D. Flies, T. Boone, H. Hsu, Y. X. Fu, S. Nagata, J. Ni, and L. Chen. Modulation of T-cell-mediated immunity in tumor and graft-versus-host disease models through the LIGHT co-stimulatory pathway. Nat. Med. 2000. 6:283
17. Wang J, Lo J C, Foster A, Yu P, Chen H M, Wang Y, Tamada K, Chen L, Fu Y X. The regulation of T cell homeostasis and autoimmunity by T cell-derived LIGHT J Clin Invest 2001. 108:1771-1780
18. Ye Q, Fraser C C, Gao W, Wang L, Busfield S J, Wang C, Qiu Y, Coyle A J, Gutierrez-Ramos J C, Hancock Modulation of LIGHT-HVEM costimulation prolongs cardiac allograft survival. J Exp Med 2002. 195:795-800
19. Tamada K, Ni J, Zhu G, Fiscella M, Teng B, van Deursen J M, Chen L. Cutting edge: selective impairment of CD8+ T cell function in mice lacking the TNF superfamily member LIGHT. J Immunol 2002. 168:4832-4835
20. Cayabyab, M., J. H. Phillips, and L. L. Lanier. CD40 preferentially costimulates activation of CD4+ T lymphocytes. J. Immunol. 1994. 152:1523
21. van Essen, D., H. Kikutani, and D. Gray. CD40 ligand-transduced co-stimulation of T cells in the development of helper function. Nature 1995. 378:620
22. Wiley, S. R., R. G. Goodwin, and C. A. Smith. Reverse signaling via CD30 ligand. J. Immunol. 1996. 157:3635
23. Cerutti, A., A. Schaffer, R. G. Goodwin, S. Shah, H. Zan, S. Ely, and P. Casali. Engagement of CD153 (CD30 ligand) by CD30+ T cells inhibits class switch DNA recombination and antibody production in human IgD+ IgM+ B cells. J. Immunol. 2000. 165:786
24. Eissner, G., S. Kirchner, H. Lindner, W. Koich, P. Janosch, M. Grell, P. Scheurich, R. Andreesen, and E. Holler. Reverse signaling through transmembrane TNF confers resistance to lipopolysaccharide in human monocytes and macrophages. J. Immunol. 2000. 164:6193
25. Chen, N. J., M. W. Huang, and S. L. Hsieh. Enhanced secretion of IFN-gamma by activated Th1 cells occurs via reverse signaling through TNF-related activation-induced cytokine. J. Immunol. 2001. 166:270
26. Suzuki, I. and P. J. Fink. Maximal proliferation of cytotoxic T lymphocytes requires reverse signaling through Fas ligand. J. Exp. Med. 1998. 187:123
27. Suzuki, I. and P. J. Fink. The dual functions of fas ligand in the regulation of peripheral CD8+ and CD4+ T cells. Proc. Natl. Acad. Sci. 2000. 97:1707
28. Suzuki, I., S. Martin, T. E. Boursalian, C. Beers, and P. J. Fink. Fas ligand costimulates the In vivo proliferation of CD8(+) T cells. J. Immunol. 2000. 165:5537
29. Wan X, Zhang J, Luo H, Shi G, Kapnik E, Kim S, Kanakaraj P, Wu J. A TNF family member LIGHT transduces costimulatory signals into human T cells. J. Immunol. 2002. 169:6813-6821

1. A tumor cell having DcR3/TR6 anchored at its surface.

2. A cell as recited in claim 1, wherein said tumor cell was transfected or transduced to express DcR3/TR6 at its surface.

3. A cell as recited in claim 1, wherein said tumor cell is malignant.

4. A cell as recited in claim 1, wherein said tumor cell is benign.

5. A cell as recited in claim 1, wherein said cell is growth inhibited.

6. A cell as recited in claim 5, wherein said growth inhibition is achieved through a treatment selected from the group consisting of a chemical treatment, irradiation, heating, freezing, and a combination thereof.

7. An immune eliciting fragment of a tumor cell as recited in claim 1.

8. A composition comprising tumor cells as recited in claim 1.

9. A composition comprising fragments as recited in claim 7.

10. A composition as recited in claim 8, further comprising an adjuvant.

11. A composition as recited in claim 8 wherein said adjuvant is BCG.

12. A recombinant vector which comprises in sequence a DNA sequence encoding a suitable promoter driving the expression of a DNA sequence encoding DcR3/TR6, and of a DNA sequence encoding a membrane anchoring peptide, and a poly A signal.

13. A method of eliciting an immune response in a patient in need thereof, comprising introducing into the patient a composition comprising tumor cells having DcR3/TR6 anchored at their surfaces or immune eliciting fragments of the cells.

14. A method as recited in claim 13, further comprising introducing a further dose of the composition.

15. A method as recited in claim 13, further comprising an simultaneous administration of a further immune therapy.

16. A method as recited in claim 13, wherein said immune therapy is selected from the group consisting of chemotherapy, radiotherapy, hormonal therapy, or a combination thereof.

17. A method as recited in claim 13, wherein the composition further comprises an adjuvant.

18. A method as recited in claim 13, wherein the adjuvant is BCG.

19. A method to inhibit the development of a tumor in a patient in need thereof, which comprises the steps of:
obtaining a cell population from a tumor mass or tissue susceptible to tumor development;
having this cell population express DcR3/TR6 on the cell membrane surface thereby obtaining a modified cell population, a fragmented cell preparation or a cell fraction comprising membrane DcR3/TR6; and
administering said modified cell population, preparation or fraction to the patient so as to elicit a stronger immune reaction towards said tumor.

20. The method as recited in claim 19, wherein said modified cell population is obtained by transfecting the cell population with a nucleic acid comprising a coding sequence of DcR3/TR6 linked to a coding sequence of a membrane anchoring peptide DNA coding sequence.
